(19) United States
(12) Patent Application Publication
(10) Pub. No.: US 2013/0123571 A1
(43) Pub. Date: May 16, 2013

(54) Systems and Methods for Streaming Psychoacoustic Therapies

(71) Applicants: Alex Doman, Ogden, UT (US); Shaye Larsen, Pleasant View, UT (US)

(72) Inventors: Alex Doman, Ogden, UT (US); Shaye Larsen, Pleasant View, UT (US)

(21) Appl. No.: 13/294,172

(22) Filed: Nov. 10, 2011

(51) Int. Cl. A61M 21/00

(52) U.S. Cl. 600/28

(57) ABSTRACT

Systems and methods for prescribing a psychoacoustic therapy to a patient or a psychoacoustic program to a user, wherein the therapy or program elements are capable of being streamed or are downloadable to the patient or user over an Internet or other network connection.

### BACKGROUND OF THE INVENTION

1. Field of the Invention

The present invention relates to the distribution of psychoacoustic therapies and related treatments to patients via streaming technologies.

2. Background and Related Art

Hearing is not a purely mechanical phenomenon of wave propagation. Rather, hearing also includes sensory and perceptual components, wherein sound waves are interpreted by the listener's brain thereby eliciting emotions, as well as physiological responses in the listener. Music therapy or psychoacoustic therapy is an allied health profession which seeks to improve the health of patients through the use of music and all of its facets, including physical, emotional, mental, social, aesthetic, and spiritual aspects of music. Music therapists primarily help patients improve their health across various domains, including cognitive functioning, motor skills, emotional and affective development, behavior and social skills, and quality of life. Music therapists use music experiences, such as singing, songwriting, listening to and discussing music, and moving to music to achieve treatment goals and objectives.

There are several concepts regarding the foundations of music therapy, including philosophies based on education, psychology, neuroscience, art/aesthetics, and music therapy itself. Accordingly, there are many different music therapy techniques from which a therapist may select when considering the best treatment for patient. Through the use of music, therapists are able to improve quality of life for the patient, as well as build self-esteem, a sense of self-worth and confidence. Therapists are further able to use music therapy to reduce stress, improve cardiovascular health (such as by using music in combination with exercise), treat heart disease and relieve other symptoms and diseases for which there are mental and physical components.

Therapy via music generally requires that the patient meet with the therapist to undergo analysis and treatment in a clinical setting. In some situations, the patient is supplied with various exercises which the patient is instructed to complete at home following a recommended therapy schedule or regimen. The patient then participates in follow-up visits with the therapist, wherein the progress of the patient is determined and changes are made to the patient's therapy, as necessary.

For some ailments, a patient may be unable or unwilling to leave the security of their home to meet with a qualified therapist. Some patients may further be unable to afford in-home therapy sessions, or have physical or mental issues which preclude in-home treatment. Accordingly, there remains a need to provide effective and convenient treatment for patients regardless of their physical or mental condition.

### BRIEF SUMMARY OF THE INVENTION

Implementation of the invention provides systems and methods for providing psychoacoustic therapies to patients. In particular, the present invention provides various systems and methods whereby a patient undergoes an initial evaluation and receives a prescribed therapy over an Internet or networked connection. Further, the patient accesses the therapy elements of the prescribed therapy by streaming or downloading the therapy elements to a computer device. As the patient progresses through the prescribed therapy, the patient's therapy is adjusted in a dynamic manner thereby providing the patient with a customized therapy that reflects the patient's progress.

Some implementations of the present invention provide a method whereby a patient completes an intake exam, whereby a baseline value is established for the patient. The baseline value is utilized to determine and prescribed a psychoacoustic therapy to the patient. In some instances, the patient accesses a therapy website to complete the intake exam, receive the prescribed therapy, and complete the various therapy elements of the prescribed therapy. For some implementations, the patient reports his or her progress through a series of questionnaires provided to the patient by a therapist or a computer software program. Additionally or alternatively, any of a variety of psychological and/or physiological measurements of the patient may be measured or determined at the time of intake or during therapy as a component or measurement of progress. The patient's updated values and/or measurements are analyzed and utilize to provide adjustments to the patient's therapy, thereby providing the patient with a customized prescribed therapy.

Some implementations of the present invention further provide a computer software program for receiving and analyzing patient values preparatory to selecting and prescribing a psychoacoustic therapy to the patient. In some instances, computer software program provides a visual feedback to the patient based upon an analysis of the patient values, for example a representative avatar. The computer software program further provides the patient with access to various therapy elements. In some instances, the computer software program further prompts the patient for updated patient values or to participate in acquisition of physiological and/or psychological measurements following completion of the prescribed therapy elements. The computer software program receives and analyzes the updated patient value to determine the effectiveness of the prescribed therapy. The computer software program then determines the effectiveness of the prescribed therapy and provides adjustments or customizations to the prescribed therapy as required to achieve the best results for the patient. In some instances, the computer software program provides the patient with access to therapy elements that are streamable or downloadable to a computer device which is accessible by the patient.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a representative computer system suitable for use with embodiments of the invention;

FIG. 2 shows a representative networked computer system suitable for use with embodiments of the invention;

FIG. 3 shows a flowchart illustrating principles associated with representative methods for streaming psychoacoustic therapies in accordance with a representative embodiment of the present invention; and

FIG. 4 shows a flowchart illustrating additional principles associated with representative methods for streaming psychoacoustic therapies in accordance with a representative embodiment of the present invention.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

Embodiments of the invention address the problem of prescribing and administering psychoacoustic therapies to individuals in a convenient and dynamic manner. Thus, embodiments of the invention provide systems, methods, and computer-readable media storing computer instructions for implementing methods for prescribing and administering music or other psychoacoustic therapies to a patient.

For convenience, the word “patient” is used herein to refer to an individual receiving psychoacoustic therapy in accordance with the principles described herein. This word, “patient,” is not intended to be limited to individuals in a traditional therapist-patient relationship, but is merely a word choice of convenience, and should be understood to apply to any individual receiving psychoacoustic therapy in accordance with the principles described herein, including, without limitation, students, employees, military personnel, family members, or any of a wide variety of end users. Therefore, unless the specific use of the word “patient” herein is specific to a traditional therapist-patient sense, the word should be read broadly as described.

Similarly, while discussion is made herein to “therapy” and “therapeutic,” it is recognized that such words have specific meanings in certain applications. Herein, these words should be given a broad interpretation as well, as systems and methods in accordance with the principles discussed herein may be used to provide download and streaming of psychoacoustic programs for purposes other than therapy as “therapy” may be defined in a particular instance, including general wellness, health maintenance, employee morale, increased learning capacity, or for any other purpose for which psychoacoustic principles are now known or may later be discovered to have applicability.

Psychoacoustics is the scientific study of sound perception. Hearing is not a purely mechanical phenomenon of wave propagation, but is also a sensory and perceptual event. In other words, when a person hears something, such as a song or rhythmic beat, that “something” arrives at the ear as mechanical sound wave traveling through the air. Once within the listener's ear, the sound wave is transformed into neural action potentials or pulses. These nerve pulses then travel to the brain where they are perceived. The perception of these nerve pulses results in physical and emotional effects on the listener. Accordingly, psychoacoustic therapy seeks to treat, change, or manipulates the physical or emotional state of a listener or patient through the use of sound.

A formalization of the process by which music generates emotional and/or physical manifestations within a patient requires an understanding of input and output variables that may be used and observed during psychoacoustic therapy. The term input variable is generally used to describe the types of audio stimulus to which the patient is exposed during a psychoacoustic therapy session. In some embodiments, input variables include specific types or genres of music. In other embodiments, input variables include structural, performance, listener, and context features of an audio stimulus, presuming that these features can, individually or in combination, produce the different emotional and/or physical manifestations described above.

Input variables may further include individual sounds or tones as produced by a singing voice or specific musical instrument. Rhythms, vocalizations, lyrics, pitches and acoustic structures may further be defined as input variables. In some embodiments, input variables further include a video stimulus which accompanies the selected acoustic stimulus. Input variables may further include physical activity by the listener, such as dance, exercise, or other movements which the listener performs in connection with listening to the audio stimulus. In some embodiments, input variables further include contextual features, such as the environment in which the patient listens to the audio stimulus. In general, input variables include any stimulus which is prescribed to a patient as part of a psychoacoustic therapy regimen.

The term output variable is generally used to describe the emotional or physical manifestations that are predicted to be produced by the input variables which have been prescribed to a patient as part of a psychoacoustic therapy. Output variables may include detectable changes in a patient's mood, behavior, emotional state, psychological state, physical condition, work performance, academic performance, stress levels, and/or any other detectable result of psychoacoustic therapy.

In some embodiments, psychoacoustic therapies are provided which comprise the prescription of one or more input variables to a patient, wherein the one or more input variables are predicted to produce one or more desired output variables in the patient. Accordingly, in some embodiments a relationship between input and output variables is known. For example, in some aspects of the present invention an input variable is selected for a patient based on a desired output variable for the patient.

The selection of the output variable for patient may be determined based upon a physical or emotional state of the patient. For example, where patient suffers from insomnia, an output variable is selected for the patient which induces or aids sleep in the patient. The input variable is thereby selected based upon the ability of the input variable to affect the desired output variable in the patient. Thus, an input variable of relaxing music may be selected were the desired output variable is an inducement of or aid to sleep in the patient.

In some embodiments, a psychoacoustic therapy is prescribed to a patient wherein the therapy comprises a plurality of input variables. In some instances, a patient meets with a therapist to undergo physical and/or psychiatric analysis to determine the desired output variables of the therapy. The therapist then prescribes various input variables to the patient, along with a regimen or schedule by which the patient is to complete or otherwise execute the input variables. In other embodiments, a psychoacoustic therapy is determined and prescribed to a patient via a computer software program, wherein the computer software program receives information from the patient and thereafter prescribes input variables to achieve one or more desired output variables.

The process by which the computer software program receives and analyzes the patient information may be achieved via a computer device connected to a network. For example, in some embodiments the patient information may be received through the patient accessing a computer program loaded onto the patient's computer device, wherein the patient's computer device and the computer software program of the present invention are operatively connected via a network, such as a local area network or the Internet. In other embodiments, the patient information is collected via a computer software program of the present invention which is loaded onto a computer device of the therapist.

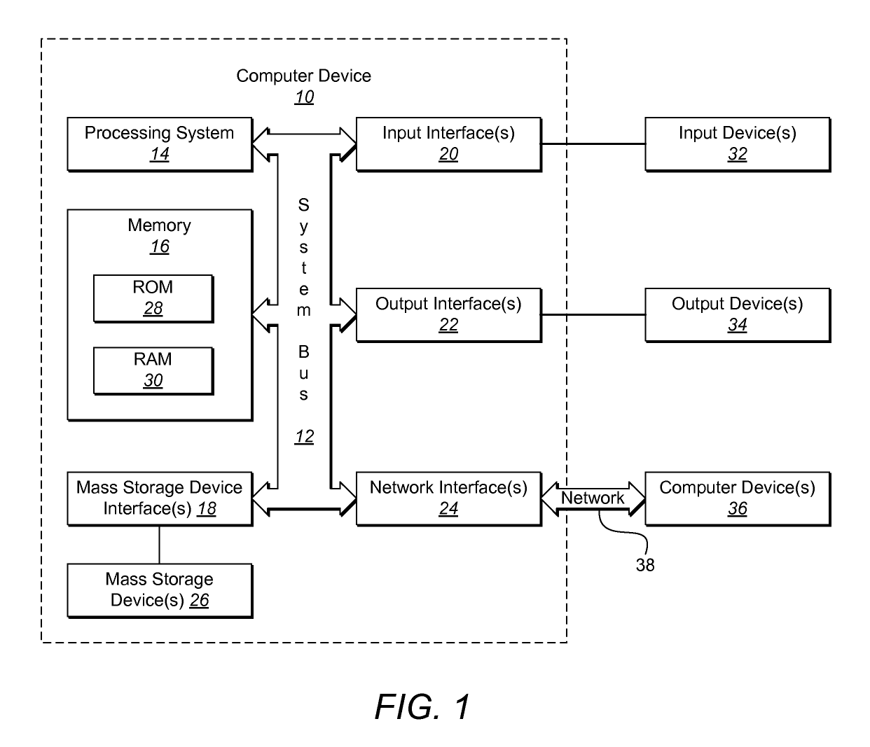

FIG. 1 and the corresponding discussion are intended to provide a general description of a suitable operating environment in which embodiments of the invention may be implemented. One skilled in the art will appreciate that embodiments of the invention may be practiced by one or more computing devices and in a variety of system configurations, including in a networked configuration. However, while the methods and processes of the present invention have proven to be particularly useful in association with a system comprising a general purpose computer, embodiments of the present invention include utilization of the methods and processes in a variety of environments, including embedded systems with general purpose processing units, digital/media signal processors (DSP/MSP), application specific integrated circuits (ASIC), stand alone electronic devices, and other such electronic environments.

Embodiments of the present invention embrace one or more computer-readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer-readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system. While embodiments of the invention embrace the use of all types of computer-readable media, certain embodiments as recited in the claims may be limited to the use of tangible, non-transitory computer-readable media, and the phrases “tangible computer-readable medium” and “non-transitory computer-readable medium” (or plural variations) used herein are intended to exclude transitory propagating signals per se.

With reference to FIG. 1, a representative system for implementing embodiments of the invention includes computer device 10, which may be a general-purpose or special-purpose computer or any of a variety of consumer electronic devices. For example, computer device 10 may be a personal computer, a notebook computer, a netbook, a tablet computer such as the iPad® manufactured by Apple or any of a variety of Andriod™-based tablet computers produced by multiple manufacturers, a personal digital assistant (“PDA”) or other hand-held device, a workstation, a minicomputer, a mainframe, a supercomputer, a multi-processor system, a network computer, a processor-based consumer electronic device, or the like.

Computer device 10 includes system bus 12, which may be configured to connect various components thereof and enables data to be exchanged between two or more components. System bus 12 may include one of a variety of bus structures including a memory bus or memory controller, a peripheral bus, or a local bus that uses any of a variety of bus architectures. Typical components connected by system bus 12 include processing system 14 and memory 16. Other components may include one or more mass storage device interfaces 18, input interfaces 20, output interfaces 22, and/or network interfaces 24, each of which will be discussed below.

Processing system 14 includes one or more processors, such as a central processor and optionally one or more other processors designed to perform a particular function or task. It is typically processing system 14 that executes the instructions provided on computer-readable media, such as on memory 16, a solid-state drive, a flash drive, a magnetic hard disk, a removable magnetic disk, a magnetic cassette, an optical disk, or from a communication connection, which may also be viewed as a computer-readable medium.

Memory 16 includes one or more computer-readable media that may be configured to include or includes thereon data or instructions for manipulating data, and may be accessed by processing system 14 through system bus 12. Memory 16 may include, for example, ROM 28, used to permanently store information, and/or RAM 30, used to temporarily store information. ROM 28 may include a basic input/output system (“BIOS”) having one or more routines that are used to establish communication, such as during start-up of computer device 10. RAM 30 may include one or more program modules, such as one or more operating systems, application programs, and/or program data.

One or more mass storage device interfaces 18 may be used to connect one or more mass storage devices 26 to system bus 12. The mass storage devices 26 may be incorporated into or may be peripheral to computer device 10 and allow computer device 10 to retain large amounts of data. Optionally, one or more of the mass storage devices 26 may be removable from computer device 10. Examples of mass storage devices include solid-state drives, flash drives, hard disk drives, magnetic disk drives, tape drives and optical disk drives. A mass storage device 26 may read from and/or write to a magnetic hard disk, a removable magnetic disk, a magnetic cassette, an optical disk, or another computer-readable medium. Mass storage devices 26 and their corresponding computer-readable media provide nonvolatile storage of data and/or executable instructions that may include one or more program modules such as an operating system, one or more application programs, other program modules, or program data. Such executable instructions are examples of program code means for implementing steps for methods disclosed herein.

One or more input interfaces 20 may be employed to enable a user to enter data and/or instructions to computer device 10 through one or more corresponding input devices 32. Examples of such input devices include a keyboard and alternate input devices, such as a mouse, trackball, touch screen, light pen, stylus, or other pointing device, a microphone, a joystick, a game pad, a satellite dish, a scanner, a camcorder, a digital camera, and the like. Similarly, examples of input interfaces 20 that may be used to connect the input devices 32 to the system bus 12 include a serial port, a parallel port, a game port, a universal serial bus (“USB”), an integrated circuit, a Firewire® (IEEE 1394), or another interface. For example, in some embodiments input interface 20 includes an application specific integrated circuit (ASIC) that is designed for a particular application. In a further embodiment, the ASIC is embedded and connects existing circuit building blocks.

One or more output interfaces 22 may be employed to connect one or more corresponding output devices 34 to system bus 12. Examples of output devices include a monitor or display screen or other electronic display, a speaker, a printer, a multi-functional peripheral, and the like. A particular output device 34 may be integrated with or peripheral to computer device 10. Examples of output interfaces include a video adapter, an audio adapter, a parallel port, and the like. Examples of electronic displays include monitors, televisions, e-ink displays, projection displays, or any other display capable of displaying changing information under the control of a computer device.

One or more network interfaces 24 enable computer device 10 to exchange information with one or more other local or remote computer devices, illustrated as computer devices 36, via a network 38 that may include hardwired and/or wireless links. Examples of network interfaces include a network adapter for connection to a local area network (“LAN”) or a modem, wireless link, or other adapter for connection to a wide area network (“WAN”), such as the Internet. The network interface 24 may be incorporated with or peripheral to computer device 10. In a networked system, accessible program modules or portions thereof may be stored in a remote memory storage device. Furthermore, in a networked system computer device 10 may participate in a distributed computing environment, such as a cloud-based computer environment, where functions or tasks are performed by a plurality of networked computer devices.

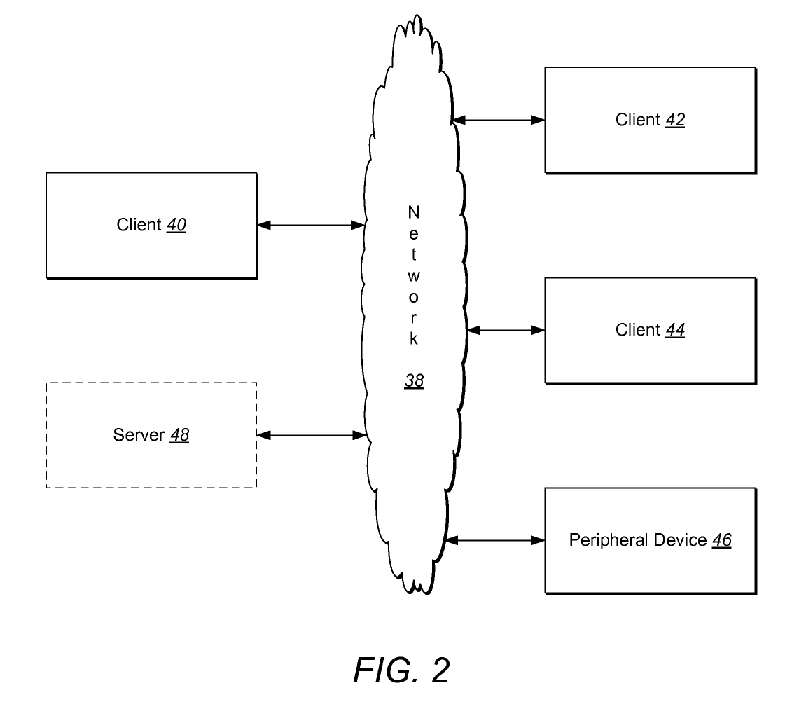

Thus, while those skilled in the art will appreciate that embodiments of the present invention may be practiced in a variety of different environments with many types of system configurations, FIG. 2 provides a representative networked system configuration that may be used in association with embodiments of the present invention. The representative system of FIG. 2 includes a computer device, illustrated as client 40, which is connected to one or more other computer devices (illustrated as client 42 and client 44) and one or more peripheral devices (illustrated as multifunctional peripheral (MFP) MFP 46) across network 38. While FIG. 2 illustrates an embodiment that includes a client 40, two additional clients, client 42 and client 44, one peripheral device, MFP 46, and optionally a server 48, which may be a print server, connected to network 38, alternative embodiments include more or fewer clients, more than one peripheral device, no peripheral devices, no server 48, and/or more than one server 48 connected to network 38. Other embodiments of the present invention include local, networked, or peer-to-peer environments where one or more computer devices may be connected to one or more local or remote peripheral devices. Moreover, embodiments in accordance with the present invention also embrace a single electronic consumer device, wireless networked environments, and/or wide area networked environments, such as the Internet.

Similarly, embodiments of the invention embrace cloud-based architectures where one or more computer functions are performed by remote computer systems and devices at the request of a local computer device. Thus, returning to FIG. 2, the client 40 may be a computer device having a limited set of hardware and/or software resources. Because the client 40 is connected to the network 38, it may be able to access hardware and/or software resources provided across the network 38 by other computer devices and resources, such as client 42, client 44, server 48, or any other resources. The client 40 may access these resources through an access program, such as a web browser, and the results of any computer functions or resources may be delivered through the access program to the user of the client 40. In such configurations, the client 40 may be any type of computer device or electronic device discussed above or known to the world of cloud computing, including traditional desktop and laptop computers, smart phones and other smart devices, tablet computers, or any other device able to provide access to remote computing resources through an access program such as a browser.

In some embodiments, a psychoacoustic therapy comprises a dynamic therapy program which is modified over time to reflect the current needs and progress of the patient. In some embodiments, a patient undergoes an initial examination process to determine the condition and needs of the patient. Following a period of time, the patient undergoes additional examination and/or reporting procedures thereby enabling the therapist or the computer software program to determine and adjust the psychoacoustic therapy regimen to achieve the desired output variables for the patient.

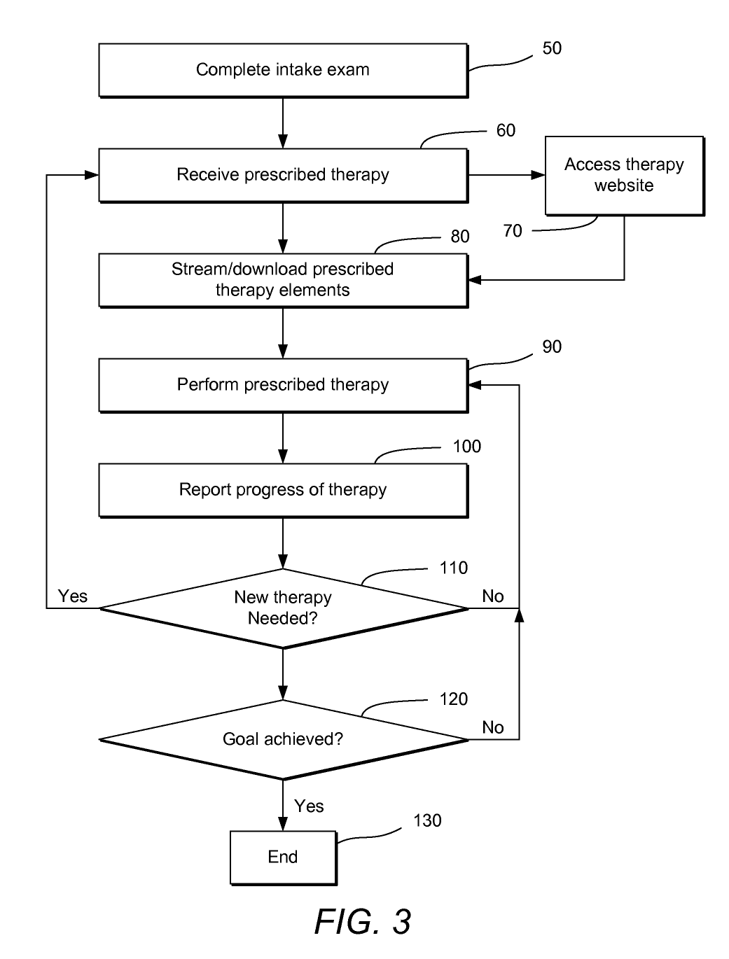

Referring now to FIG. 3, a process by which the patient receives and undergoes a psychoacoustic therapy is shown. In some embodiments, the patient initiates the process of receiving a psychoacoustic therapy by completing an intake exam 50. The intake exam generally comprises various questions which are designed to determine and evaluate the psychological and physical condition of the patient. In some embodiments, the intake exam comprises a plurality of Likert scale questions, wherein the patient is requested to assign a personal value to each category and/or question, true-false questions, multiple choice questions, or other analytical tools. In some embodiments, the intake exam further comprises physical analysis, such as measurements of the patient's heart rate, voice analysis, temperature, and body weight. The intake exam may include standardized questions which are designed for the general population, or may include non-standardized questions which are designed for specific groups of patients. For example, in some embodiments non-standardized questions are provided which target a specific race, gender, sex, age group, profession, or medical condition of the patient.

Following completion of the intake exam, a psychoacoustic therapy is prescribed to the patient 60. In some embodiments, a therapist reviews the results of the intake exam and determines the patient's psychoacoustic therapy. In other embodiments, a decision engine of a computer software program selects various input variables based upon the results of the intake exam, as described in further detail below. The process by which the patient receives the prescribed therapy may include a step whereby the patient accesses the intake exam and the prescribed therapy via a website 70. For example, in some embodiments a membership-based website is provided whereby a patient registers with the website and is thereafter provided with access to the intake exam and the subsequently prescribed psychoacoustic therapy. In other embodiments, the patient is provided with an executable software program which the patient downloads onto the patient's personal computer device. The patient then accesses the executable software program to complete the intake exam and/or access the prescribed psychoacoustic therapy. Further, in some embodiments the process by which the patient completes the intake exam 50 and receives the prescribed therapy 60 comprises a mixture of web-based and computer software-based applications.

In some embodiments, the prescribed input variables or therapy elements may include items which achieve sound brain training in the patient. For example, these elements may include various audio pieces, music-based or otherwise, which have been determined to achieve certain output variables in patients. These elements may further include video media which provide images to the patient which have been determined to achieve certain output variables. Multimedia therapy elements may also be prescribed, wherein the multimedia content comprises audio and video components which have been determined to achieve certain output variables in patients. Therapeutic elements may further include interactive software programs which present the patient with information which has been determined to achieve certain desired output variables.

As described above, input variables of the present invention may include various digital forms of media which may be remotely streamed or downloaded by the patient 80, such as to the user's mobile electronic device. In some embodiments, the prescribed therapy comprises a plurality of Internet Web addresses, whereby the patient streams, downloads, or otherwise receives the prescribed therapy elements by clicking or otherwise selecting the Internet Web addresses. The Internet Web addresses provide the patient with direct access to the prescribed therapy elements. For example, in some embodiments an Internet Web address is linked to a streamable or downloadable therapy element. In other embodiments, an Internet Web address is linked to an Internet resource whereby the patient is able to receive the prescribed therapy element via streaming over the Internet.

In addition to receiving prescribed therapy elements, the patient is further prescribed a regimen or schedule by which the patient is to complete the various therapy elements 90. The regimen or schedule for the prescribed psychoacoustic therapy may include any length and/or frequency of exposure to the therapy elements as may be determined to achieve the desired output variables in the patient. In some embodiments, the patient performs the prescribed therapy by logging into a website, wherein the website presents the patient with a schedule of activities or other therapeutic elements for completion by the patient. For example, the therapy website may require the patient to log into the website each day to receive a therapy element prescribed for that day.

The website may include a calendar by which the patient monitors his or her progress in completing the prescribed therapeutic regimen. Further, the website may include a progress bar or other means whereby the patient monitors his or her progress. For example, in some embodiments the patient is prompted to report the progress of the patient's therapy 100. The process or mechanism by which the patient reports progress may simply include a system of reporting whereby the patient checks or marks a box which indicates that a prescribed therapy element has been completed. The process or mechanism may further include an interactive questionnaire whereby the patient is tested to determine the effectiveness of the therapy following completion of a prescribed therapy element. The process or mechanism may further include measurement or determination of physiological and/or psychological conditions of the patient, similar to the measurement or determination performed as part of the intake exam.

In some embodiments, the therapist or computer software program analyzes the patient's progress to determine the effectiveness of the prescribed therapy and regimen. The therapist or computer software program then makes a determination as to whether the patient could benefit from an adjustment in the prescribed psychoacoustic therapy 110. In some instances, the progress of the patient indicates that the patient is benefiting from the prescribed therapy, and therefore does not require a new or adjusted therapy. Rather, the patient is instructed to continue performing the previously prescribed therapy 90. In other instances, the progress of the patient indicates that the prescribed therapy is insufficient to achieve the desired output variables. Accordingly, a new, adjusted or customized therapy or therapy regimen is prescribed to the patient 60.

In some embodiments, the progress of the patient indicates that the therapeutic goals for the patient have been achieved 120. At the point in which the desired output variables are achieved, the therapy for the patient ends 130, or may optionally be replaced by a maintenance therapy program designed to maintain the desired output variables. Where the desired output variables for the patient have not been achieved, the patient continues to perform the prescribed therapy 90, which may be modified as necessary as discussed above.

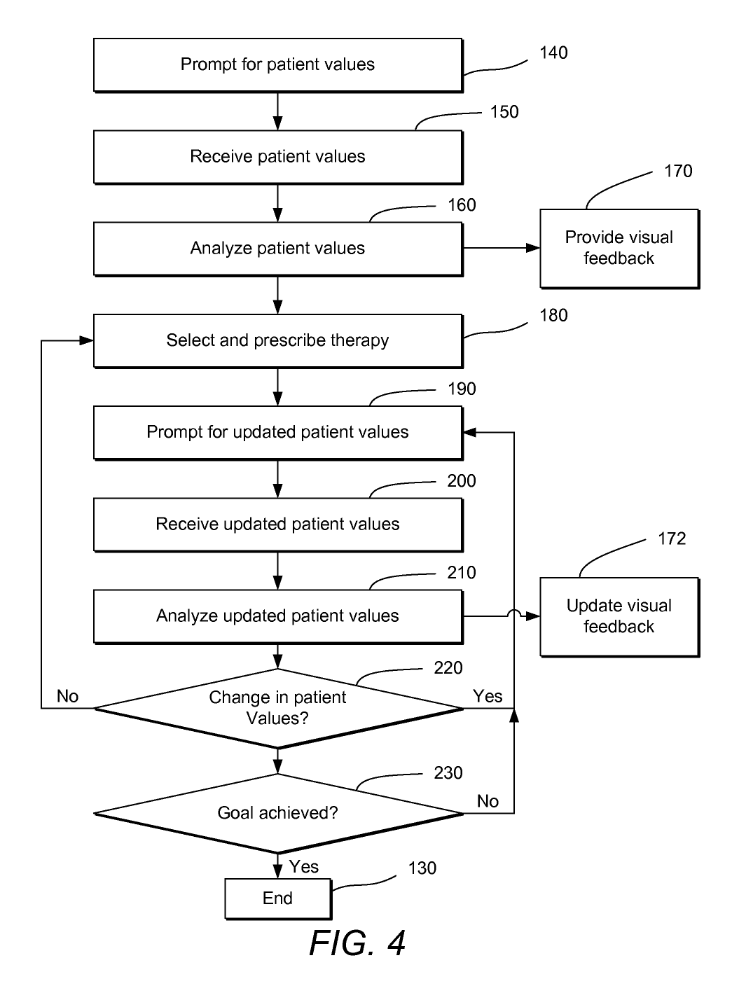

Referring now to FIG. 4, a process by which a computer software program prescribes, tracks, and updates a psychoacoustic therapy for a patient is shown. In some embodiments, the computer software program is accessed by the patient via a website and a web browser. In other embodiments, the computer software program is accessed by a computer device onto which the computer software program is loaded. Further, in some embodiments the computer software program is accessed via a mobile computer device, such as a smart phone, a tablet, a personal digital assistant, a media player, or other processor-based device capable of communicating with a wireless network. For example, in some embodiments the computer software program is a mobile application which the patient downloads onto the patient's mobile device.

The computer software program is generally provided as a means to assist the patient in receiving, accessing, and performing the various input variables or therapy elements which have been prescribed to the patient. In some embodiments, the computer software program provides a prompt to receive various patient values 140. Patient values may include information to identify the patient, or a condition of the patient for which therapy is required. In some embodiments, the computer software program provides the patient with an intake examination in the form of various prompts. The prompts may include a checkbox, or series of checkboxes from which the patient is requested to select a choice which most accurately represents an opinion, feeling, characteristic, symptom, or other value of the patient. The prompts may further include a text field in which the patient provides a written answer in response to a question. Further still, the prompts may comprise a Likert scale, wherein the patient is prompted to select a value on the scale which most accurately represents the patient. The prompts may also provoke or direct the patient to provide physiological and/or psychological inputs to the computer software program.

The computer software program receives the patient values 150 and then performs an analysis of the patient values 160. The process by which the software program analyzes the patient values is to compare the patient values to a standardized set of scores which have been established based on the effectiveness of an input variable to achieve a desired output variable. In some embodiments, the patient values are categorized as educational data, physiological parameters data, emotional data, and psychological data. Thus, the computer software program may assign a score to the patient based upon a categorization of the patient values. The computer software program may then use the patient's score to assist in selecting and prescribing a therapy to the patient.

The specific mechanism and procedure by which the computer software program analyzes the patient values may be achieved by any methodology or processing/analysis methods known in the art. Accordingly, the systems and methods of the present invention are not limited to any specific method of analysis for determining an effective therapy for a patient.

In some embodiments, the computer software program further comprises a feature for providing the patient with a visual representation of the patient's received values 170. For example, in some embodiments the computer software program analyzes the patient values 160 and thereafter renders a visual representation of the patient in the form of a representative avatar of the patient. The patient is therefore provided with a visual feedback 170 which indicates an overall physical, emotional, and psychological condition of the patient. For example, where the patient values indicate that the patient is overweight, depressed, and emotionally stressed, the representative avatar of the patient would comprise visual characteristics which mimic or otherwise represent the patient's condition.

In some embodiments, the computer software program further comprises a feature for providing the patient with a visual representation of the patient's results based upon fulfillment of a prescribed psychoacoustic therapy. For example, in some embodiments the computer software program analyzes the patient values 160 and thereafter determines a desired physical, emotional, and psychological condition of the patient. The computer software program then renders a prophetic visual representation of the patient in the form of an avatar which shows or otherwise represents the predicted physical, emotional, and psychological condition of the patient following completion of the prescribed psychoacoustic therapy. Thus, the prophetic avatar provides the patient with a visual goal to serve as an incentive for completing the prescribed therapy. In some embodiments, the computer software program further provides the patient with a current representative avatar and a prophetic avatar to demonstrate to the patient the potential effectiveness of the prescribed therapy based on a comparison of the two avatars and/or a comparison of the two avatars to the original avatar.

Following analysis of the patient values 160, the computer software program selects and prescribes a psychoacoustic therapy to the patient 180. As previously discussed, the psychoacoustic therapy includes various input variables which may be accessed by the patient over an Internet or other network connection. In some embodiments, the input variables comprise streaming audio, video and/or software which the patient accesses over the Internet. In other embodiments, the input variables comprise one or more streamable and/or downloadable files, thereby enabling the patient to stream and/or download the input variables and access the contents of the files offline.

In some embodiments, the computer software program selects the input variables from a library of therapy element files. The therapy element files are selected based upon their ability to achieve a desired output variable in a patient when included as part of a psychoacoustic therapy regimen. Therapy element files may include any type of audio, video or multimedia format. In some embodiments, therapy element files are categorized based upon their intended or predicted effect. For example, in some embodiments therapy element files are categorized as psychoacoustic files, compliance files, patient files, student files, educational files, and customer files. Accordingly, the computer software program may select and prescribed various therapy element files based upon a category or score of the patient which is determined from the patient values.

In some embodiments, the computer software program provides the patient with a plurality of therapy elements and a schedule by which the patient is to complete the therapy elements. The patient accesses the therapy elements by streaming or downloading the therapy element file, as discussed previously. In some instances, the patient accesses the therapy element files by logging into a therapy website which comprises a library of therapy element files. In some instances, the therapy website further comprises the patient's prescribed therapy and regimen, such that the website automatically selects and provides the patient with the specific therapy element file based upon the website having access to the patient's prescribed therapeutic regimen.

In some embodiments, the computer software program provides a prompt to the patient requesting updated patient values 190. The updated patient values 190 may include values similar to those discussed above, and may be obtained in similar fashion. In some instances, a patient completes a prescribed therapy element file and is thereafter prompted to complete a questionnaire. The computer software program receives and records the patient values from the questionnaire and updates the patient's profile to include the updated patient values 200. The computer software program then analyzes the updated patient values to determine the progress of the patient's prescribed psychoacoustic therapy 210.

In some embodiments, the computer software program analyzes the updated patient values 210 and thereafter updates the visual feedback of the patient 172 to reflect physical, emotional, and psychological changes to the patient. For example, where the visual feedback is an avatar, the physical, emotional, and psychological changes to the patient are represented as visual modifications to the appearance of the avatar. In some embodiments, the color, shape, density, facial expression, body shape, posture, size or other physical characteristic of the avatar is modified to represent a change in the patient based upon the updated patient values. Accordingly, the patient receives real-time visual feedback based upon the patient's completion of the prescribed therapeutic elements.

Where the computer software program detects a change in the patient values, a determination is made regarding whether the change in the patient values represents progress in the patient based upon the prescribed psychoacoustic therapy 220. If the change in the patient values indicates that the patient is progressing, the computer software program continues to prompt the patient for updated patient values 190. If the change in patient values indicates that the patient is not progressing, or if there is no detectable change in the patient values, the computer software program selects and prescribes a new therapy 180. Thus, the prescribed therapy is dynamic in nature and may be customized by the computer software program to achieve a desired output variable for the patient. As discussed above, therapy may also be changed and modified based on the patient's positive progress.

In some embodiments, the computer software program monitors the frequency and length of exposure of the patient to the various prescribed therapy elements, and uses this information to adjust or customize the patient's prescribed therapy. For example, where a patient fails to complete the prescribed therapeutic regimen, or does not completes the prescribed therapy elements within a recommended period of time, the computer software program may adjust the patient's therapy to include additional therapy elements to compensate for the patient's lack of diligence in completing the prescribed therapy. Further, the computer software program may prompt the patient with additional questionnaire questions to determine the affect of the patient's lack of diligence in completing the prescribed therapeutic regimen. The computer software program may then use the patient values to customize the patient's therapy in order to compensate for any deficiency caused by the patient's behavior.

The computer software program further analyzes the updated patient values to determine if the patient's goals have been achieved 230. At the point in which the desired output variables are achieved, the computer software program indicates to the patient that the therapy is complete 240. Where the desired output variables for the patient have not been achieved, the computer software program continues to prompt the patient for updated patient values 190 as the patient continues to complete the prescribed psychoacoustic therapy.

In some embodiments, the computer software program further comprises predictive modeling capabilities, wherein the program learns the patterns of the patient's progress based upon various factors and data of the patient which are collected by the computer software program. Thus, the computer software program becomes self-informed as the patient progresses through the prescribed therapy. Accordingly, the computer software program may adjust the prescribed therapy and regimen based upon the program's predictive modeling of the patient's behaviors and progress, thereby providing a customized therapy for the patient.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A computer-implemented method for prescribing a psychoacoustic therapy, the method comprising:
prompting a patient for an initial patient value;
receiving and analyzing the initial patient value;
selecting and prescribing a psychoacoustic therapy comprising streamable or downloadable therapy elements;
providing the streamable or downloadable therapy elements over a computer network;
prompting the patient for an updated patient value;
receiving and analyzing the updated patient value; and
adjusting the psychoacoustic therapy based upon the updated patient values.

2. The method of claim 1, further comprising a step for providing a visual feedback based upon at least one of the initial patient value and the updated patient value.

3. The method of claim 1, wherein the streamable or downloadable therapy elements comprise at least one of an audio file, a video file, a software file, and a multimedia file.

4. The method of claim 1, wherein providing the streamable or downloadable therapy elements over a computer network comprises streaming at least one of the streamable or downloadable therapy elements over the computer network.

5. The method of claim 1, wherein providing the streamable or downloadable therapy elements over a computer network comprises downloading at least one of the streamable or downloadable therapy elements over the computer network for later consumption.

6. The method of claim 1, wherein adjusting the psychoacoustic therapy comprises selecting and prescribing a modified psychoacoustic therapy comprising streamable or downloadable therapy elements that differ from the streamable or downloadable therapy elements of the psychoacoustic therapy initially selected and prescribed.

7. The method of claim 6, wherein the psychoacoustic therapy is automatically and iteratively adjusted over a period of time based on the patient's progress as determined by updated patient values iteratively obtained over the period of time.

8. A computer-implemented method for providing a psychoacoustic program, the method comprising:
prompting a user for an initial input value relevant to determining a psychoacoustic program element designed to obtain a desired psychoacoustic output variable;
receiving and analyzing the initial input value;
selecting and providing a psychoacoustic program comprising streamable or downloadable program elements;
providing the streamable or downloadable program elements over a computer network;
prompting the user for an updated input value;
receiving and analyzing the updated input value; and
adjusting the psychoacoustic program based upon the updated input values.

9. The method of claim 8, wherein the initial input value and the updated input value comprise input values related to one of a physiological, intellectual, and mental state of the user.

10. The method of claim 8, further comprising a step for providing a visual feedback based upon at least one of the initial input value and the updated input value.

11. The method of claim 8, wherein the streamable or downloadable program elements comprise at least one of an audio file, a video file, a software file, and a multimedia file.

12. The method of claim 8, wherein providing the streamable or downloadable program elements over a computer network comprises streaming at least one of the streamable or downloadable program elements over the computer network.

13. The method of claim 8, wherein providing the streamable or downloadable program elements over a computer network comprises downloading at least one of the streamable or downloadable program elements over the computer network for later consumption.

14. The method of claim 8, wherein adjusting the psychoacoustic program comprises selecting and prescribing a modified psychoacoustic program comprising streamable or downloadable program elements that differ from the streamable or downloadable program elements of the psychoacoustic program initially selected and provided.

15. The method of claim 14, wherein the psychoacoustic program is automatically and iteratively adjusted over a period of time based on the user's progress as determined by updated input values iteratively obtained over the period of time.

16. The method of claim 8, wherein the initial input value is obtained through an intake exam.

17. The method of claim 8, wherein the psychoacoustic program is continually and automatically modified and updated over a period of time based on user compliance with the psychoacoustic program and further based on updated input values of the user updated over the period of time.

18. A non-transitory computer-readable medium storing computer program instructions for implementing a method for prescribing a psychoacoustic therapy, the method comprising:
prompting a patient for an initial patient value;
receiving and analyzing the initial patient value;
selecting and prescribing a psychoacoustic therapy comprising streamable or downloadable therapy elements;
prompting the patient for an updated patient value;
receiving and analyzing the updated patient value; and
adjusting the psychoacoustic therapy based upon the updated patient values.

19. The computer-readable medium of claim 18, wherein providing the streamable or downloadable therapy elements over a computer network comprises streaming at least one of the streamable or downloadable therapy elements over the computer network.

20. The computer-readable medium of claim 18, wherein the method further comprises iteratively repeating the steps of prompting the patient for an updated patient value, receiving and analyzing the updated patient value; and adjusting the psychoacoustic therapy, whereby the psychoacoustic therapy is continually and automatically modified and updated over a period of time based on the updated patient values as they are updated over the period of time.
